(19) United States
(12) Patent Application Publication
(10) Pub. No.: US 2011/0065675 A1
(43) Pub. Date: Mar. 17, 2011

(54) AZO DYE RELATED SMALL MOLECULE MODULATORS OF PROTEIN-PROTEIN INTERACTIONS

(75) Inventors: Peter Buchwald, Miami, FL (US); Emilio Margolles-Clark, Miami, FL (US); Norma S. Kenyon, Miami, FL (US); Camillo Ricordi, Miami, FL (US)

(73) Assignee: University of Miami, Miami, FL (US)

(21) Appl. No.: 12/991,079

(22) PCT Filed: May 5, 2009
(86) PCT No.: PCT/US09/42831

§ 371 Date: Nov. 4, 2010

Related U.S. Application Data
(60) Provisional application No. 61/071,525, filed on May 5, 2008.

(51) Int. Cl. A61K 31/655; A61K 31/185; C12N 5/071; A61P 35/00; A61P 3/10; A61P 29/00; A61P 19/04; A61P 3/00; A61P 17/00; A61P 17/06; A61P 37/02; A61P 37/06; A61P 25/28; A61P 9/10

(52) U.S. Cl. 514/150; 514/577; 435/375

(57) ABSTRACT

Azo dyes and suramin-related small molecules are effective in inhibiting the CD40/CD154 protein-protein interaction, an important co-stimulatory interaction involved in the activation of immune responses mediated by T- and B-cells. The compounds were found to be active as indicated by their IC₅₀ values both in a cell-free binding assay and in the inhibition of CD154-induced B-cell proliferation assay. The compounds may be used as therapeutic compounds for treatment of diseases and disorders related to immune or inflammatory responses. Methods of inhibiting the CD40/CD154 protein-protein interaction and treating diseases and disorders related to immune or inflammatory responses are described.

### CROSS-REFERENCE TO RELATED APPLICATION

This application claims priority to U.S. Provisional Application No. 61/071,525 filed May 5, 2008, the entire contents of which are hereby incorporated by reference.

### BACKGROUND

### Small Molecule Protein-Protein Interaction Inhibitors (PPII)

Protein-protein interactions (PPI) play key roles in many biological processes; hence, they offer attractive opportunities for therapeutic interventions. The identification and/or development of small molecules capable to modulate PPIs is of obvious interest. However, this was thought to be difficult mainly because of the lack of well-defined binding pockets that are present on the traditional targets of most existing drugs (GPCRs, ion channels, enzymes) and because of the relatively large protein surface areas involved in these PPIs (Arkin et al., “Small-molecule inhibitors of protein-protein interactions: progressing towards the dream,” Nat. Rev. Drug Discov., vol. 3, pp. 301-317, 2004); hence, it was not pursued for a long time. Recently, it has become clear that small molecules can interfere with these interactions in certain cases quite effectively, and promising progress has been made along these lines in the identification of small molecule inhibitors for PPIs such as B7/CD28, B7/CTLA4, IL-2/IL-2R, LFA1/ICAM, β-catenin/Tcf3&4 and others including G protein subunits (Arkin et al., “Small-molecule inhibitors of protein-protein interactions: progressing towards the dream,” Nat. Rev. Drug Discov., vol. 3, pp. 301-317, 2004; Wells et al., “Reaching for high-hanging fruit in drug discovery at protein-protein interfaces,” Nature, vol. 450, pp. 1001-1009, 2007).

### CD40/CD154 Co-Stimulatory Blockade

Blocking of the costimulatory protein-protein interaction is one of the most actively investigated pathways to mitigate immune responses in transplant patients and even in autoimmune diseases. According to the current knowledge, T cell activation is thought to require two signals (or three, if growth signals are included): engagement of the T cell receptor (TCR) with the MHC-peptide complex (signal 1) and ligation of costimulatory molecules on T cells with their respective ligands on antigen-presenting cells (APCs) (signal 2). T cells receiving signal 1 and positive costimulation undergo proliferation, cytokine production, and further differentiate into effector cells. Even if the underlying mechanisms are not entirely understood, it is generally believed that antigen recognition in the absence of costimulation may alter the immune response and ultimately lead to tolerance. Two primary costimulatory molecules on T cells have been extensively studied: 1) CD28, whose cognate ligands are CD80 (B7-1) and CD86 (B7-2) on APCs, and 2) CD154 (CD40 ligand), whose interactions with CD40 on APCs bidirectionally activate both T cells and APCs (Gao et al., “Negative T cell costimulation and islet tolerance,” Diabetes Metab. Res. Rev., vol. 19, pp. 179-185, 2003; Larsen et al., “A new look at blockade of T-cell costimulation: a therapeutic strategy for long-term maintenance immunosuppression,” Am. J. Transplant., vol. 6, pp. 876-883, 2006; Vincenti et al. “T cell costimulation: a rational target in the therapeutic armamentarium for autoimmune diseases and transplantation,” Annu. Rev. Med., vol. 58, pp. 347-358, 2007; Weaver et al., “Costimulation blockade: towards clinical application,” Front Biosci, vol. 13, pp. 2120-2139, 2008).

CD154 (CD40L, gp39, TRAP) is a 34-39 kDa type II membrane glycoprotein, a member of the tumor necrosis factor (TNF) family of cell surface interaction molecules, mainly expressed on activated (CD4⁺) but not resting T cells, and also on activated B cells, activated platelets, and other cells (Grewal et al. “The role of CD40 ligand in costimulation and T-cell activation,” Immunol. Rev., vol. 153, pp. 85-106, 1996; van Kooten et al. “CD40-CD40 ligand,” J. Leukoc. Biol., vol 67, pp. 2-17, 2000; Schönbeck et al., “The CD40/CD154 receptor/ligand dyad,” Cell Mol. Life. Sci., vol. 58, pp. 4-43, 2001).). Its receptor, CD40 is a 45-50 kDa type I membrane protein expressed on primary B cells, monocytes, macrophages, dendritic cells, and even pancreatic duct and β-cells (Barbé-Tuana et al. “CD40-CD40 ligand interaction activates proinflammatory pathways in pancreatic islets,” Diabetes, vol. 55, pp. 2437-2445, 2006).) CD40 and CD154 form trimers when they interact, and this interaction induces B-cell activation, differentiation, clonal expansion, isotype switching, affinity maturation, germinal center formation, generation of long-lived plasma cells, and activation of dendritic cells (van Kooten et al., “J. CD40-CD40 ligand,” J. Leukoc. Biol., vol. 67, pp. 2-17, 2000; Quezada et al., “CD40/CD154 interactions at the interface of tolerance and immunity,” Annu. Rev. Immunol., vol. 22, pp. 307-328, 2004; Daoussis et al., “Targeting CD40L: a promising therapeutic approach,” Clin. Diagn. Lab. Immunol., vol. 11, pp. 635-641, 2004; Allen, et al. “Therapeutic peptidomimetic strategies for autoimmune diseases: costimulation blockade,” J. Pept. Res., vol. 65, pp. 591-604, 2005). The interaction of CD154 on T-cells and its receptor CD40 on B-cells is essential for lymphocyte signaling leading to T cell-dependent B cell proliferation, immunoglobulin class switching, and B-cell maturation. Mutations of CD154 expressed on T-lymphocytes are known to result in X-linked hyper-IgM syndrome (XHIGM), a primary immunodeficiency characterized by an inability to produce immunoglobulins of the IgG, IgA, and IgE isotypes (Thusberg et al., “The structural basis of hyper IgM deficiency—CD40L mutations,” Protein Eng. Des. Sel., vol. 20, pp. 133-141, 2007). Blocking of CD154 (CD40L, gp39, TRAP) from interacting with its receptor, CD40 (Grewal et al., “The role of CD40 ligand in costimulation and T-cell activation,” Immunol. Rev., vol. 153, pp. 85-106, 1996; van Kooten et al., “CD40-CD40 ligand,” J. Leukoc. Biol., vol. 67, pp. 2-17, 2000; Schönbeck et al., “The CD40/CD154 receptor/ligand dyad,” Cell Mol. Life. Sci., vol. 58, pp. 4-43, 2001) is known to be a highly effective means by which to abrogate autoimmune diseases and induce transplantation tolerance (Quezada et al. “CD40/CD154 interactions at the interface of tolerance and immunity,” Annu. Rev. Immunol., vol. 22, pp. 307-328, 2004; Daoussis et al., “Targeting CD40L: a promising therapeutic approach,” Clin. Diagn. Lab. Immunol., vol. 11, pp. 635-641, 2004; Burkly, L. C., “CD40 pathway blockade as an approach to immunotherapy,” Adv. Exp. Med. Biol., vol. 489, pp. 135-152, 2001). Because this attack is directed mainly toward activated T cells, where CD154 is primarily expressed, a more specific immune suppression is expected, and because costimulation is suppressed, such treatment is more likely to lead to altering of the immune response and long-term tolerance.

It has been shown that transplantation of an adequate number of functional islets combined with an anti-CD154 monoclonal antibody (mAb) monotherapy consistently allowed for allogeneic islet engraftment and long-term insulin independence in various animal models (Molano et al. “Prolonged islet graft survival in NOD mice by blockade of the CD40-CD154 pathway of T-cell costimulation,” Diabetes, vol. 50, pp. 270-276, 2001) including nonhuman primate nonhuman primate (NHP) models (Kenyon et al., “Long-term survival and function of intrahepatic islet allografts in rhesus monkeys treated with humanized anti-CD154,” Proc. Natl. Acad. Sci. U.S.A., vol. 96, pp. 8132-8137, 1999; Kenyon et al., “Long-term survival and function of intrahepatic islet allografts in baboons treated with humanized anti-CD154,” Diabetes, vol. 48, pp. 1473-1481, 1999). Anti-CD154 mAbs are also essential components of different immunosuppressive regimens that allow long-term islet allograft function (Koulmanda et al., “Prolonged survival of allogeneic islets in cynomolgus monkeys after short-term anti-CD154-based therapy: nonimmunologic graft failure?” Am. J. Transplant., vol. 6, pp. 687-696, 2006),) possibly even in certain xenografts (e.g., porcine to NHP) (Cardona et al., “Long-term survival of neonatal porcine islets in nonhuman primates by targeting costimulation pathways,” Nat. Med., vol. 12, pp. 304-306, 2006; Cardona et al. “Engraftment of adult porcine islet xenografts in diabetic nonhuman primates through targeting of costimulation pathways,” Am. J. Transplant., vol. 7, pp. 2260-2268, 2007).

However, clinical trials of the corresponding humanized antibody (ruplizumab, hu5c8) for systemic lupus erythematosus (SLE), multiple sclerosis (MS), and kidney transplant have been halted because of thrombolitic side effects (Kawai et al, “Thromboembolic complications after treatment with monoclonal antibody against CD40 ligand,” Nat. Med., vol. 6, p. 114, 2000) and development is no longer supported (Couzin, J., “Drug discovery. Magnificent obsession,” Science, vol. 307, pp. 1712-1715, 2005). Activated platelets express CD154; however, in platelet-rich plasma, the 5c8 antibody itself did not induce platelet aggregation per se and did not significantly affect maximal aggregation; it has been suggested that CD154 expression produced by physiological or pathophysiological platelet activation can sustain a pro-aggregatory effect of the antibody by a mechanism involving the mAb Fc domain (Mirabet et al., “Platelet pro-aggregatory effects of CD40L monoclonal antibody,” Mol. Immunol., vol. 45, pp. 937-944, 2008). It is also encouraging that a recently identified cyclic heptapetide (CLPTRHMAC) capable of blocking the CD40/CD154 interaction did not prime human platelet activation and aggregation in in vitro platelet activation studies contrary to the anti-CD154 mAb tested (Deambrosis et al., “Inhibition of CD40-CD154 costimulatory pathway by a cyclic peptide targeting CD154,” J. Mol. Med., vol. 87, pp. 181-197, 2009).

Therefore, the targeting of the CD40/CD154 pathway with small molecule inhibitors is of particular interest for transplant recipients in general and for pancreatic islet transplant recipients in particular. Furthermore, the CD40/CD154 co-stimulatory interactions also seems one of the most promising targets to prevent generation of type 1 diabetes (T1D) as an autoimmune disease (Balasa et al., “CD40 ligand-CD40 interactions are necessary for the initiation of insulitis and diabetes in nonobese diabetic mice,” J. Immunol., vol. 150, pp. 4620-4627, 1997; Bour-Jordan et al., “Costimulation controls diabetes by altering the balance of pathogenic and regulatory T cells,” J. Clin. Invest., vol. 114, pp. 979-987, 2004), and antagonizing the effects of CD154 (and its soluble form) might provide other therapeutic benefits as well. Ligation of CD40 is known to mediate a variety of immune and inflammatory responses, such as the expression of adhesion molecules, cytokines, matrix-degrading enzymes, prothrombotic activities, and apoptotic mediators (Schönbeck et al., “The CD40/CD154 receptor/ligand dyad,” Cell Mol. Life Sci., vol. 58, pp. 4-43, 2001). Consequently, inhibition of the CD40 signaling can be beneficial in pathogenic processes of chronic inflammatory diseases, such as autoimmune diseases, neurodegenerative disorders, graft-versus-host disease, cancer, and atherosclerosis.

Small molecule CD40/CD154 inhibitors have been described by Zheng et al. (U.S. Pat. No. 7,173,046). The most effective compound class described by Zheng et al. has activity characterized only as <50 μM. There are four groups of scientific publications describing peptide CD40/CD154 inhibitors; the activity of the corresponding peptides are as follows: large cyclic peptides, MW ˜2500, with trimeric symmetry with estimated IC₅₀ in the 50-100 nM range (Fournel et al., “C₃-symmetric peptide scaffolds are functional mimetics of trimeric CD40L,” Nat. Chem. Biol., vol. 1, pp. 377-382, 2005; Wieckowski et al., “Cooperativity in the interaction of synthetic CD40L mimetics with CD40 and its implication in cell signaling,” Biochemistry, vol. 46, pp. 3482-3493, 2007; Trouche et al., “Small multivalent architectures mimicking homotrimers of the TNF superfamily member CD40L: delineating the relationship between structure and effector function,” J. Am. Chem. Soc., vol. 129, pp. 13480-13492, 2007; Habib et al., “Cutting edge: small molecule CD40 ligand mimetics promote control of parasitemia and enhance T cells producing IFN-gamma during experimental Trypanosoma cruzi infection,” J. Immunol., vol. 178, pp. 6700-6704, 2007), two end-group-blocked peptides with estimated IC₅₀ around 100 μM (Allen et al., “Therapeutic peptidomimetic strategies for autoimmune diseases: costimulation blockade,” J. Pept. Res., vol. 65, pp. 591-604, 2005), cyclic heptapetides with activities in the 10-50 μM range (Deambrosis et al., “Inhibition of CD40-CD154 costimulatory pathway by a cyclic peptide targeting CD154,” J. Mol. Med., vol. 87, pp. 181-197, 2009), and three recombinant phage proteins with very low activity (≈100 mM) (Kitagawa et al., “Identification of three novel peptides that inhibit CD40-CD154 interaction,” Mod. Rheumatol., vol. 15, pp. 423-426, 2005).

### Azo Dyes and Suramin-Related Small Molecules

Azo dyes are usually aryl azo compounds containing the Ar—N═N—Ar′ functional group. They are usually stable, crystalline species and are available in large structural variety as they are commonly used in various dyeing or coloring applications (Hunger K, Ed., Industrial Dyes. Chemistry, Properties, Applications. Weinheim: Wiley-VCH, 2003). The majority of azo dyes, including food and textile dyes, have median lethal dose (LD₅₀) values in the 250-2,000 mg/kg range, and a number of them have been investigated at various times for possible therapeutic activities. For example, direct red 75 (chlorazol fast pink, Sirius rose BB) has been used for its anticoagulant activity as it was shown to inhibit the thrombin-fibrinogen reaction (Modell, W., “Chlorazol fast pink BKS as an anti-coagulant,” Science, vol. 89, pp. 349-350, 1939; Merskey et al., “The anticoagulant action of chlorazol fast pink,” Br. J. Haematol., vol. 2, pp. 276-282, 1956).

Another more recent example, FP-21399, a bis(disulphonaphthalene)-azo compound selected from a screening program of Fuji compounds originally developed for photographic use for the potential treatment of HIV infections (as a possible inhibitor of the gp120-mediated fusion that also showed some PPII activity for the CD4-gp120 binding) has reached clinical trials (Ono et al., “FP-21399 blocks HIV envelope protein-mediated membrane fusion and concentrates in lymph nodes,” Nat. Biotechnol., vol. 15, pp. 343-348, 1997), and, for example, i.v. doses of 3 mg/kg once weekly provided plasma levels expected to be therapeutically adequate (Cₘₐₓ of 30-40 μg/mL, t_{1/2β}=4 h, t_{1/2γ}=40 h) with no serious side effects (but a transient, dose-dependent appearance of drug- or metabolite-related color in the urine and skin) (Dezube et al, “A fusion inhibitor (FP-21399) for the treatment of human immunodeficiency virus infection: a phase I study,” J. Infect. Dis., vol. 182, pp. 607-610, 2000).

Suramin is a known P2 (ATP/UTP purine receptor) antagonist (IC₅₀≈5-10 μM) (Ralevic et al., “Receptors for purines and pyrimidines,” Pharmacol. Rev., vol. 50, pp. 413-492, 1998) that is also a known inhibitor of the binding of a range of tumor growth factors, and has various other biological activities as well (Voogd et al. “Recent research on the biological activity of suramin,” Pharmacol. Rev., vol. 45, pp. 177-203, 1993). It is approved for the prophylactic treatment of African sleeping sickness (trypanosomiasis) and river blindness (onchocerciasis), infections caused by parasites, and it has been investigated for antiviral (HIV) and antitumor activity (Kaur et al. “Suramin's development: what did we learn?” Invest. New Drugs, vol. 20, pp. 209-219, 2002).

### SUMMARY

In one aspect, the invention deals with methods and uses of azo dyes and suramin-related small molecules to inhibit the CD40/CD154 costimulatory protein-protein interaction.

Readily available azo dyes and suramin-related small molecules were found to be effective in inhibiting the CD40/CD154 costimulatory protein-protein interaction, which is involved in the activation of immune responses mediated by T- and B-cells. Compounds may be active in the 0.5-50 μM range as indicated by their IC₅₀ values both in cell-free binding assays and in the inhibition of CD154-induced B-cell proliferation assay (Table 1).

Significant CD40/CD154 binding inhibitory activity following standard dose-response curves was found (Table 1) in a cell-free binding assay using both human (Example 1) and murine (Example 2) proteins. These compounds may also show corresponding activity in inhibiting the CD154-induced human B-cell proliferation assay (Example 3).

Therefore, the activity of these compounds in blocking the CD40/CD154 costimulatory interaction is particularly promising. Most of these compounds are well-known azo-dyes, a large structural variety of such compounds are easily available, and many are relatively non-toxic. These compounds do not appear to have been considered for such purposes despite their obvious ability to bind well to various proteins.

In another aspect, the invention relates to the use of azo dyes and suramin-related small molecules as therapeutic agents. For example, the compounds may be used as immune-modulators, tolerance inducing agents, or anti-inflammatory agents. The azo dyes and suramin-related small molecules may be used for the manufacture of medicaments or pharmaceutical compositions for the treatment of diseases or disorders involving the immune system or for immune suppression or to induce tolerance in recipients of non-autologous organ or cell transplants.

In another aspect, the invention provides methods of inhibiting CD40/CD154 protein-protein interactions by exposing the proteins to azo dyes or suramin-related small molecules. In some cases, the method involves administering azo dyes or suramin-related small molecules to a subject in need of treatment for a disease or disorder involving the immune system. Therefore, the invention also includes methods of treating diseases and disorders involving the immune system by administering an effective amount of an azo dye or suramin-related small molecule to a subject in need of treatment.

### BRIEF DESCRIPTION OF THE DRAWINGS

FIG. 1. Concentration-dependent inhibition of the human CD40/CD154 binding by representative compounds of the present invention with an anti-CD154 antibody (mAb) as a positive control and tartrazine (TZ) as a negative control. Data (symbols) are average±SD (normalized to percent binding) from multiple independent experiments with duplicates or triplicates for each condition and fitted with a standard log(inhibitor) vs. response model (lines).

FIG. 2. Concentration dependent inhibition of the mouse CD40/CD154 binding by representative compounds of the present invention with an anti-CD154 antibody (mAb) as a positive control and tartrazine (TZ) as a negative control. Data (symbols) are average±SD (normalized to percent binding) from multiple independent experiments with duplicates or triplicates for each condition and fitted with a standard log(inhibitor) vs. response model (lines).

FIG. 3. Assessment of the binding partner (CD40 vs. CD154) by quantification of the amount of protein bound after incubations of the test compounds with one of the proteins, CD154 (bottom) and CD40 (top), and addition of the other only after a wash-out. Most compounds bind to CD154 and not to CD40. Data (symbols) are average±SD (normalized to percent binding) from multiple independent experiments with duplicates or triplicates for each condition and fitted with a standard log(inhibitor) vs. response model (lines).

FIG. 4. Dose-dependent inhibition of CD154-induced human CD19⁺ B-cell proliferation by representative compounds of the present invention with the anti-CD154 antibody (mAb) as positive control. Data are average±SD from at least two independent experiments with triplicates for each condition.

FIG. 5. Concentration-dependent inhibition of the human TNF-R1/TNFα binding by representative compounds of the present invention with an anti-TNFα antibody (mAb) as a positive control. Data (symbols) are average±SD (normalized to percent binding) from multiple independent experiments with duplicates or triplicates for each condition and fitted with a standard log(inhibitor) vs. response model (lines).

FIG. 6. Structures of representative compounds.

FIG. 7. Structures of representative compounds.

### DETAILED DESCRIPTION

Embodiments of the invention include the use of an azo dye or suramin-related small molecule to inhibit the CD40/CD154 protein-protein interaction.

For purposes of this application, azo dyes include compounds containing an aryl azo (Ar—N═N—Ar′) structure that are commonly used for dyeing or coloring purposes. In certain embodiments, the compounds may also contain one or more acidic functionalities (such as a sulfonic or carboxylic acid). Such acidic functionalities may be present in a salt form where an acidic hydrogen has been replaced with a non-hydrogen cation. In further embodiments, the azo dyes are mono or polysulfonylated, meaning the structures include at least one sulfonic acid moiety, which may have an acidic hydrogen or other counter-ion.

Extensive examples of azo dyes may be found, for example in Industrial Dyes. Chemistry, Properties, Applications (Hunger K, Ed., Weinheim: Wiley-VCH, 2003), and the Colour Index (C.I.) online (www.colour-index.org).

Examples of suitable counter-ions are described, for example, by Remington et al. (Remington: The Science and Practice of Pharmacy, 21^{st} Ed., 2005; Remington: The Science and Practice of Pharmacy (20th ed.), ed. A. R. Gennaro, Lippincott Williams & Wilkins, 2000), which is incorporated by reference in its entirety. For example, the counter-ion may be sodium, potassium or other alkali metal cations, alkaline earth metal cations, or organic cations.

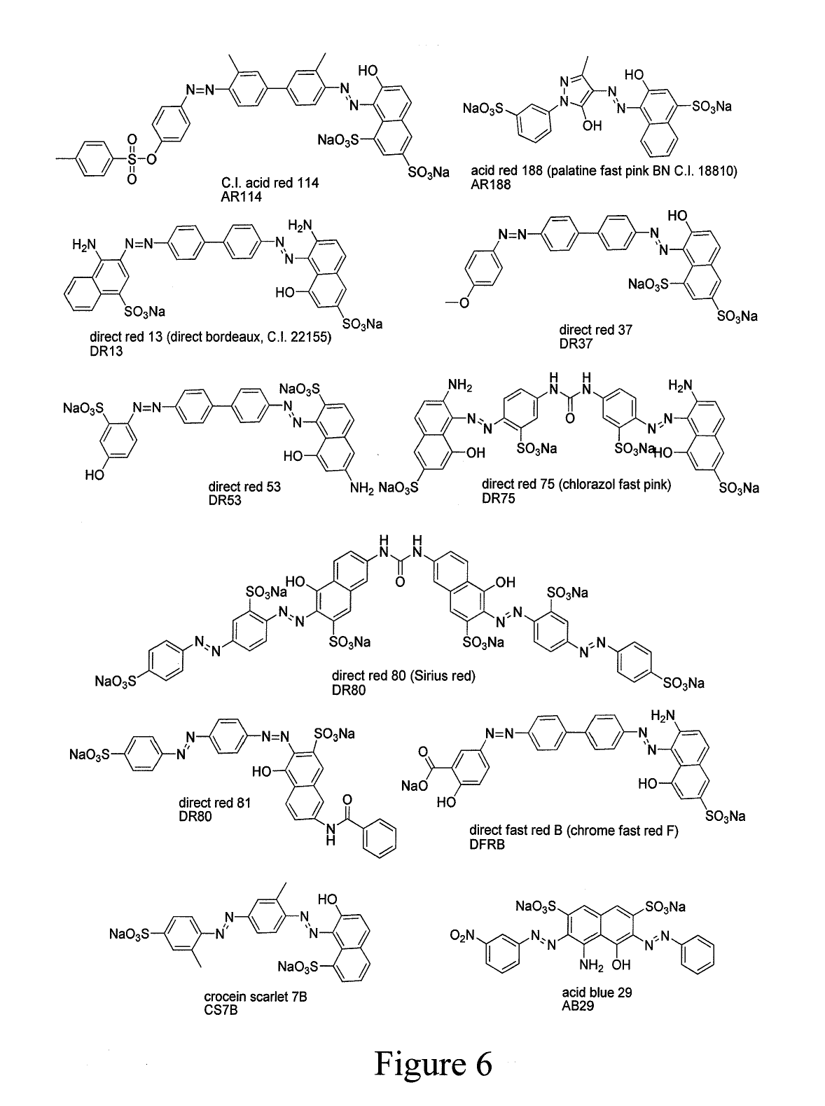

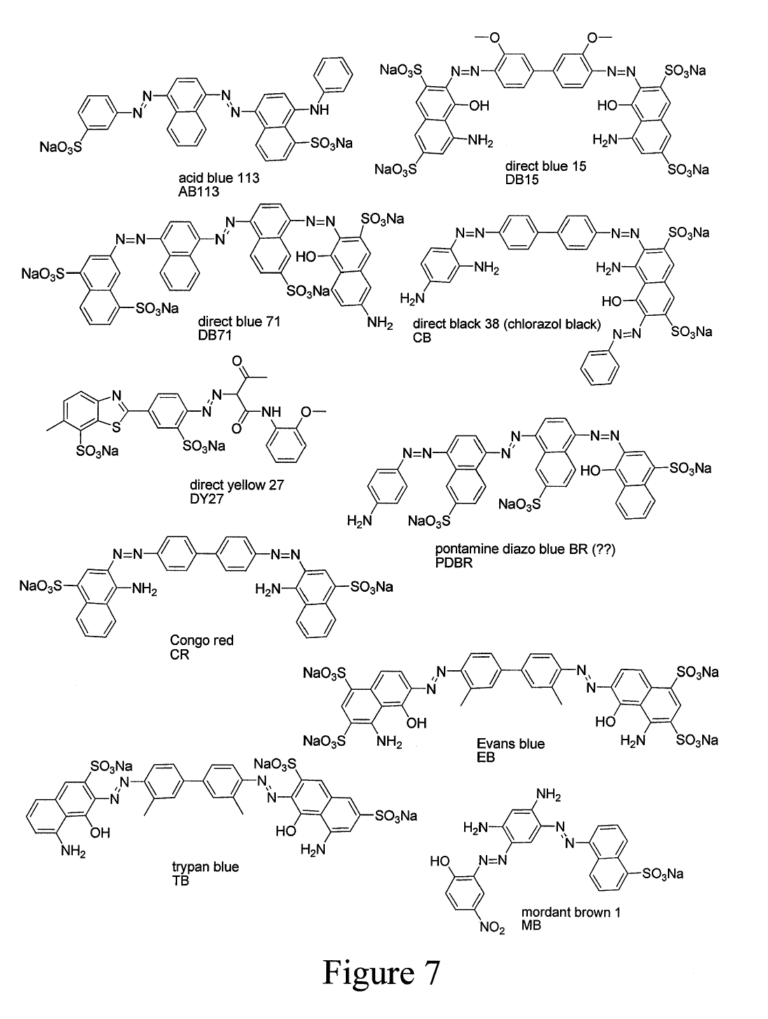

Examples of azo dyes that inhibit the CD40/CD154 interaction include C.I. acid red 114 (AR114),), acid red 188 (palatine fast pink BN C.I.18810) (AR188), direct red 13 (direct bordeaux, C.I. 22155) (DR13), direct red 37 (DR37), direct red 53 (DR53), direct red 75 (chlorazol fast pink) (DR75), direct red 80 (Picrosirius red) (DR80), and direct red 81 (DR81), direct fast red B (chrome fast red F) (DFRB), crocein scarlet 7B (CR7B), acid blue 29 (AB29), acid blue 113 (AB113), direct blue 15 (DB15), direct blue 71 (DB71), direct black 38 (chlorazol black) (CB), direct yellow 27 (DY27), direct blue 120 (pontamine diazo blue BR) (PDBR), Congo red (CR), trypan blue (TB), Evans blue (EB), and mordant brown 1 (MB). Structures of these compounds are shown in FIG. 6 and FIG. 7. The structures are shown as sodium salts, but in all cases, the sodium ion may be replaced with hydrogen or another suitable counter-ion.

For purposes of this application, suramin-related small molecules are compounds structurally related to suramin that maintain essentially the same structural framework (i.e., ring network), but in which (i) methyl or other simple alkyl substituent as well as halo (—F, —Cl, —Br, —I) on the aromatic rings are added, removed, or moved at arbitrary positions along the rings (ii) one or more aromatic benzene rings are replaced by naphthyl or by heterocyclic aromatic rings such as pyridine, pyrazine, indole, imidazole, pyrazole, oxazole, thiazole, furan, thiophene, and others or the connections to other rings are moved to other relative positions (i.e., ortho-, meta-, or para-positions); and (iii) aromatic sulfonic acid moieties (—SO₃M) are added, removed, or moved at arbitrary positions along the rings as known to those skilled in the arts. Therefore, the suramin-related small molecules according to the present invention are expressed by the structure shown below, including suramin itself:

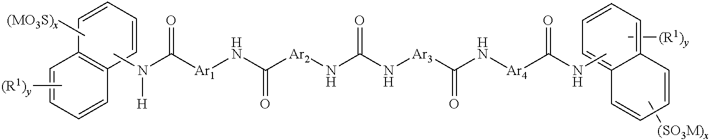

Where Ar₁, Ar₂, Ar₃ and Ar₄ are each phenyl, naphthyl, or heteroaryl, where each may be substituted by one or more R¹ or SO₃M; R¹ is simple alkyl, halogen, —F, —Cl, —Br, or —I; M is H or a mono-cationic counter ion; x is 1, 2, 3, or 4; y is 0, 1, 2, 3, or 4. It is understood that each —SO₃M or R₁ moiety may be located at any position of the Ar or terminal naphthalene rings, and that more than one —SO₃M or R₁ moiety may be present on each Ar or terminal naphthalene rings.

The term “simple alkyl” as used herein means straight-chain, branched, or cyclic C₁-C₆ hydrocarbons which are completely saturated and hybrids thereof such as (cycloalkyl)alkyl. Examples of simple alkyl substituents include methyl, ethyl, propyl (including n-propyl (ⁿPr), iso-propyl (ⁱPr), and cyclopropyl (^{c}Pr)), butyl (including n-butyl (n-Bu, ⁿBu), isobutyl (i-Bu, ¹Bu), sec-butyl (s-Bu, ^{s}Bu), tert-butyl (t-Bu, ^{t}Bu), or cyclobutyl (c-Bu, ^{c}Bu)), and so forth.

The term “heteroaryl” refers to heteroaromatic ring groups having five to fourteen members, preferably five to ten, in which one or more ring carbons, preferably one to four, are each replaced by a heteroatom such as N, O, or S. Examples of heteroaryl rings include furanyl, imidazolyl, isoxazolyl, oxadiazolyl, oxazolyl, pyrrolyl, pyrazolyl, pyridyl, pyrimidyl, pyrazinyl, pyridazinyl, thiazolyl, thiazolyl, tetrazolyl, triazolyl, thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, or benzoisoxazolyl. The term “heteroaryl” also refers to rings that are optionally substituted by one or more R¹ or SO₃M, defined above. The term “heteroaryl” may be used interchangeably with the term “heteroaryl ring” or the term “heteroaromatic”. Preferred heteroaryl groups include pyridinyl, pyrazinyl, indolyl, imidazolyl, pyrazolyl, oxazolyl, thiazolyl, furanyl, and thienyl.

In some embodiments, the suramin-related small molecules of the present invention are expressed by the structure shown below, including suramin itself:

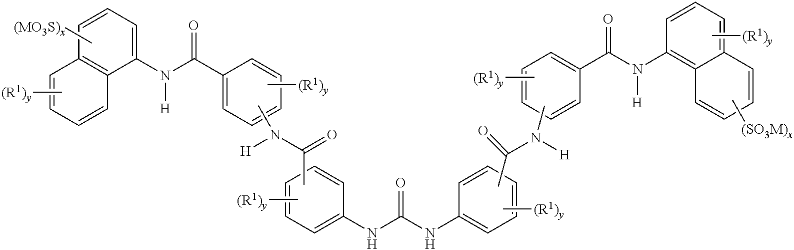

where R¹ is simple alkyl, halogen, —F, —Cl, —Br, or —I; M is H or a mono-cationic counter ion; x is 1, 2, 3, or 4; and y is 0, 1, 2, 3 or 4. It is understood that each —SO₃M or R¹ moiety may be located at any position of the naphthalene ring.

Specific examples of suramin-related small molecules include suramin itself and NF279 shown below, with the understanding that one or more acidic hydrogens in each structure may be replaced by a suitable counter-ion.

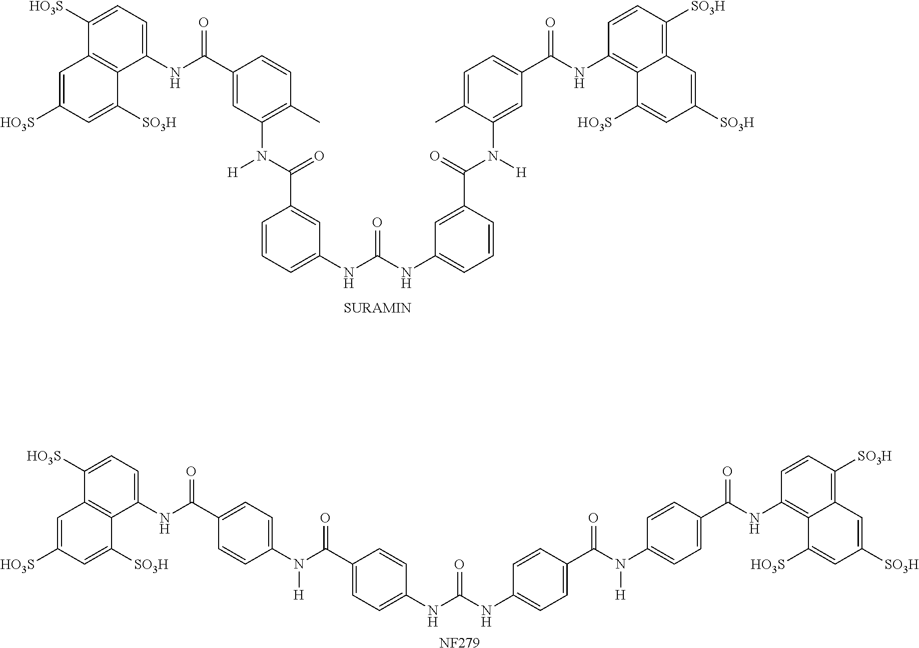

In certain embodiments, the azo dye or suramin-related small molecule is selected from the group consisting of C.I. acid red 114, acid red 188, direct red 13, direct red 37, direct red 53, direct red 75, direct red 80, and direct red 81, direct fast red B, crocein scarlet 7B, acid blue 29, acid blue 113, direct blue 15, direct blue 71, direct blue 120, direct black 38, direct yellow 27, Congo red, trypan blue, Evans blue, mordant brown 1 as well as suramin and NF279.

In certain embodiments, the azo dye or suramin-related small molecule may be used in an ex vivo culture of an organ or cell transplant, before transplantation into a subject, to improve cell or organ survival during culture and ameliorate inflammatory and immune responses following transplantation. In some specific embodiments, the transplanted cells may be pancreatic islet cells or stem cells. In some cases, cells would be exposed to the azo dye or suramin-related small molecule only during their culture, before transplanting them into the recipient. For example, the cells may be exposed to the azo dye or suramin-related small molecule by using the azo dye or suramin-related small molecule as an additional ingredient of the culture media.

In certain embodiments, the azo dyes and suramin-related small molecules may be used in a therapeutic setting for the treatment of diseases and disorders regulated by CD40/CD154 protein-protein interaction, as immune modulators, tolerance inducing agents or anti-inflammatory agents, or for immune suppression or tolerance induction in a recipient of a nonautologous organ or cell transplant. For instance, azo dyes and suramin-related small molecules may be used for the manufacture of medicaments for treatment of diseases and disorders related to the immune system.

Diseases and disorders regulated by CD40/CD154 protein-protein interaction include diseases and disorders involving the immune system, such as chronic inflammatory diseases, autoimmune diseases, neurodegenerative disorders, graft-versus-host disease, cancer, atherosclerosis, or the rejection of nonautologous organ or cell transplants. Specific examples of autoimmune diseases include systemic lupus erythematosus (SLE), multiple sclerosis (MS), type 1 (juvenile) diabetes, rheumatoid arthritis, mixed connective tissue disease (MCTD), Celiac disease, Crohn's disease, ulcerative colitis, Grave's disease, Sjögren's syndrome, dermatomyositis, psoriasis, scleroderma, polymyositis, vasculitis, Wegener's granulomatosis, and alopecia areata.

In certain embodiments, the azo dye or suramin-related small molecule is used as an immune modulator, tolerance inducing agent, or anti-inflammatory agent. Other embodiments involve the use of azo dyes and suramin-related small molecules for immune suppression or tolerance induction in a recipient of a nonautologous organ or cell transplant. In specific embodiments, the transplant is a pancreatic islet transplant.

Further embodiments include the use of azo dyes and suramin-related small molecules for the treatment of diseases or disorders involving the immune system. Specific diseases or disorders involving the immune system include chronic inflammatory diseases, autoimmune diseases, neurodegenerative disorders, graft-versus-host disease, cancer, atherosclerosis, or the rejection of nonautologous organ or cell transplants. Specific examples of autoimmune diseases include systemic lupus erythematosus (SLE), multiple sclerosis (MS), type 1 (juvenile) diabetes, rheumatoid arthritis, mixed connective tissue disease (MCTD), Celiac disease, Crohn's disease, ulcerative colitis, Grave's disease, Sjögren's syndrome, dermatomyositis, psoriasis, scleroderma, polymyositis, vasculitis, Wegener's granulomatosis, and alopecia areata.

In certain embodiments, the azo dyes and suramin-related small molecules may be used for investigational purposes. Certain embodiments include ELISA-type screening procedures such as, but not limited to those described in Examples 1, 2, 3, or 5 using the azo dyes and suramin-related small molecules to identify stuctural scaffolds required to inhibit costimulatory or other receptor-ligand type protein-protein interactions of interest. Such protein-protein interactions include, but are not limited to, TNF-R1/TNF-α (human tumor necrosis factor-alfa to its receptor), CD80(B7)/CD28, CD80(B7)/CD152(CTLA4), CD86(B7-2)/CD28, CD86/CD152, CD27/CD70, CD137(4-1BB)/4-1BBL, HVEM/LIGHT(CD258), CD30/CD30L, GITR/GITRL, BAFF-R(CD268)/BAFF(CD257), RANK(CD265)/RANKL(CD254), OX40(CD134)/OX40L(CD252), ICOS(CD278)/ICOS-L(CD175), IL-2/IL-2R (interleukin-2 with its receptor), and LFA1/ICAM. For example, the ELISA-type assays are calibrated and then used to determine the inhibitory activity of selected azo dyes. The most active compounds identified are then used to select similar and other structures likely to be active, whose inhibitory actifities are determined, and the process is iteratively repeated as needed. Comparison of the structures of the active and inactive compounds can be used to establish the structural elements (e.g. ring structures, and functional moieties) required for activity and to derive structure activity relationships.

Other embodiments include methods of inhibiting CD40/CD154 protein-protein interactions comprising exposing the proteins to azo dyes or suramin-related small molecules. In certain embodiments, the azo dye or suramin-related small molecule is selected from the group consisting of C.I. acid red 114, acid red 188, direct red 13, direct red 37, direct red 53, direct red 75, direct red 80, and direct red 81, direct fast red B, crocein scarlet 7B, acid blue 29, acid blue 113, direct blue 15, direct blue 71, direct blue 120, direct black 38, direct yellow 27, Congo red, trypan blue, Evans blue, mordant brown 1 as well as suramin and NF279.

In certain embodiments, the method may be practiced in vitro, in cell culture, or in a human or non-human (e.g., mammalian) subject.

In certain embodiments, the step of exposing the protein to an azo dye or suramin-related small molecule in the method may take the form of ex vivo culture of an organ or cell transplant in the presence of azo dyes or suramin-related small molecules before transplantation into a subject. This may function to improve survival during culture and ameliorate inflammatory and immune responses following transplantation. In some specific embodiments, the transplanted cells may be pancreatic islet cells or stem cells. In some cases, cells would be exposed to the azo dy or suramin-related small molecule only during their culture, before transplanting them into the recipient. For example, the cells may be exposed to the azo dye or suramin-related small molecule by using the azo dye or suramin-related small molecule as an additional ingredient of the culture media.

In certain embodiments, the step of exposing the protein to an azo dye or suramin-related small molecule in the method may take the form of administering an effective amount of an azo dye or suramin-related small molecule to a subject in need of treatment for a disease or disorder mediated by the CD40/CD154 protein-protein interaction. In this case, an effective amount is an amount sufficient to measurably inhibit the CD40/CD154 protein-protein interaction in the subject. Preferably, symptoms of said disease or disorder will be alleviated.

In certain embodiments, therefore, the method may be used to treat a disease or disorder mediated by the CD40/CD154 protein-protein interaction. Such diseases and disorders include diseases and disorders involving the immune system. In such cases, an effective amount is an amount sufficient to alleviate at least one symptom of said disease. Specific diseases or disorders involving the immune system include chronic inflammatory diseases, autoimmune diseases, neurodegenerative disorders, graft-versus-host disease, cancer, atherosclerosis. or the rejection of nonautologous organ or cell transplants. Examples of specific autoimmune diseases systemic lupus erythematosus (SLE), multiple sclerosis (MS), type 1 (juvenile) diabetes, rheumatoid arthritis, mixed connective tissue disease (MCTD), Celiac disease, Crohn's disease, ulcerative colitis, Grave's disease, Sjögren's syndrome, dermatomyositis, psoriasis, scleroderma, polymyositis, vasculitis, Wegener's granulomatosis, and alopecia areata.

Other embodiments include methods of modulating the immune system, inducing tolerance, or reducing inflammation to a subject in need comprising administering an effective amount of an azo dye or suramin-related small molecule to a subject. In this case, an effective amount is an amount sufficient to detectably inhibit the CD40/CD154 protein-protein interaction in the subject, for instance, by detectably inducing tolerance, reducing inflammation, or otherwise beneficially modulating the immune system.

Other embodiments include a method of treating a disease or disorder mediated through the CD40/CD154 pathway comprising administering an effective amount of an azo dye or suramin-related small molecule to a subject in need of treatment. In certain embodiments, the disease or disorder is a disease or disorder involving the immune system. For instance, diseases and disorders which involve activation of immune responses mediated by T- and B-cells. Diseases or disorders involving the immune system include chronic inflammatory diseases, autoimmune diseases, neurodegenerative disorders, graft-versus-host disease, cancer, atherosclerosis or the rejection of nonautologous organ or cell transplants. Specific examples of autoimmune diseases include systemic lupus erythematosus (SLE), multiple sclerosis (MS), type 1 (juvenile) diabetes, rheumatoid arthritis, mixed connective tissue disease (MCTD), Celiac disease, Crohn's disease, ulcerative colitis, Grave's disease, Sjögren's syndrome, dermatomyositis, psoriasis, scleroderma, polymyositis, vasculitis, Wegener's granulomatosis, and alopecia areata. In such cases, an effect amount of an azo dye or suramin-related small molecule is an amount sufficient to alleviate at least one symptom of said disease or disorder.

Therapeutic applicability requires an acceptable degree of selectivity/specificity for the protein-protein interaction of interest. In certain embodiments, the azo dyes and suramin-related small molecules may be selective for CD40/CD154 interaction inhibition, indicated by a difference in activity compared with other protein-protein binding assays.

In certain embodiments, the activity of the azo dyes and suramin-related small molecules of the present invention may be 30 or more times less active in another TNF family receptor-ligand or other protein-protein binding assay. In other words, the IC₅₀ values of the azo dyes and suramin-related small molecules of the present invention may be 30 times higher in, for example, TNF-R1/TNFα binding assay compared with a corresponding CD40/CD154 binding assay. This is especially relevant since CD154 is a member of the TNF superfamily. For example, a compound may have a median inhibitory concentration (IC₅₀) in a human CD40/CD154 assay of 1 μM and a median inhibitory concentration in a human TNF-R1/TNFα of only 30 μM. Therefore, the compound would be 30 times less active in inhibiting TNF-R1/TNFα a binding than in inhibiting CD40/CD154 binding. In some embodiments, the activity may be 100 times less in the TNF-R1/TNFα binding assay.

In some embodiments, the azo dye or suramin-related small molecule has an IC₅₀ of 50 μM or less in a human CD40/CD154 binding inhibition assay.

In some embodiments, the azo dyes or suramin-related small molecules may be administered as a pharmaceutically acceptable salt, or in combination with a pharmaceutically acceptable carrier or excipient. Certain embodiments therefore include pharmaceutical compositions of certain azo dyes or suramin-related small molecules. Pharmaceutical compositions may comprise an azo dye or suramin-related small molecule and a pharmaceutically acceptable carrier or excipient. Specific embodiments include pharmaceutical compositions comprising an azo dye or suramin-related small molecule selected from the group consisting of C.I. acid red 114, acid red 188, direct red 13, direct red 53, direct red 80, direct red 81, direct fast red B, crocein scarlet 7B, acid blue 29, acid blue 113, direct blue 15, direct blue 71, direct black 38, direct yellow 27, direct blue 120, Congo red, trypan blue, Evans blue, mordant brown 1, suramin, and NF279; and a pharmaceutically acceptable carrier or excipient.

In one or more embodiments, the azo dyes and suramin-related small molecules employed in the present invention may be made into pharmaceutical compositions by combination with appropriate pharmaceutically acceptable excipients, carriers, or diluents, and may be formulated into preparations in solid, semi-solid, liquid, or gaseous forms such as tablets, capsules, powders, granules, ointments, solutions, suppositories, injections, inhalants, and aerosols in the usual ways for their respective route of administration. The following methods and excipients are merely exemplary and are in no way limiting.

In pharmaceutical dosage forms, the azo dyes or suramin-related small molecules employed in the present invention may be used in the form of their pharmaceutically acceptable salts, and also may be used alone or in appropriate association, as well as in combination with other pharmaceutically active compounds.

In the case of oral preparations, the azo dyes or suramin-related small molecules may be used alone or in combination with appropriate additives to make tablets, powders, granules or capsules, e.g., with conventional additives such as lactose, mannitol, corn starch, or potato starch; with binders such as crystalline cellulose, cellulose derivatives, acacia, corn starch, or gelatins; with disintegrators such as corn starch, potato starch, or sodium carboxymethylcellulose; with lubricants such as talc or magnesium stearate; and, if desired, with diluents, buffering agents, moistening agents, preservatives, and flavoring agents.

Furthermore, the azo dyes or suramin-related small molecules employed in the present invention may be made into suppositories by mixing with a variety of bases such as emulsifying bases or water-soluble bases.

The azo dyes or suramin-related small molecules employed in the present invention may be formulated into preparations for injections by dissolving, suspending, or emulsifying them in an aqueous or non-aqueous solvent, such as vegetable oil, synthetic aliphatic acid glycerides, esters of higher aliphatic acids or propylene glycol; and, if desired, with conventional additives such as solubilizers, isotonic agents, suspending agents, emulsifying agents, stabilizers, and preservatives.

In the cases of inhalations or aerosol preparations, the azo dyes or suramin-related small molecules employed in the invention in the form of a liquid or minute powder may be combined in an aerosol container with gas or liquid spraying agents, and, if desired, together with conventional adjuvants such as humidifying agents. They may also be formulated as pharmaceuticals for non-pressured preparations such as in a nebulizer or an atomizer.

The amount of the azo dyes or suramin-related small molecules employed in the present invention to be administered varies according to the degree of the disease or disorder encountered, and the stages of the disease. A suitable dosage is that which will result in effecting a detectable alleviation of at least one symptom of said disease or disorder. The preferred dosage is that amount sufficient to render a host asymptomatic to the particular disease or disorder.

Unit dosage forms for oral administration (such as syrups, elixirs, and suspensions) wherein each dosage unit, e.g., teaspoonful, tablespoonful, contains a predetermined amount of the azo dyes or suramin-related small molecules employed in the present invention, can be dissolved or suspended in a pharmaceutically acceptable carrier, such as Sterile Water for Injection, USP, or by normal saline.

The azo dyes or suramin-related small molecules employed in the present invention can be utilized in aerosol formulation to be administered via inhalation. The azo dye derivatives employed in the present invention can be formulated into compositions with pressurized acceptable propellants such as dichlorodifluoromethane, propane, nitrogen, and the like.

The term “unit dosage form,” as used herein, refers to physically discrete units suitable as unitary dosages for human and animal subjects, each unit containing a predetermined quantity of the azo dyes or suramin-related small molecules in an amount sufficient to produce the desired effect in association with a pharmaceutically acceptable, diluent, carrier, or vehicle. The specifications for the novel unit dosage forms of the present invention depend on the particular compound employed and the effect to be achieved, and the pharmacodynamics associated with each compound in the host.

The pharmaceutically acceptable excipients, for example, vehicles, adjuvants, carriers, or diluents, are readily available to the public.

Any necessary adjustments in dose can be readily made to meet the severity of the disease or disorder and adjusted accordingly by the skilled practitioner.

The examples disclosed below are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

### EXAMPLES

### Example 1

### Human CD40/CD154 Binding Inhibition Assay

A 96-well plate-based cell-free in vitro binding inhibition assay, which is a modification of an assay described in U.S. Pat. No. 7,173,046, incorporated by reference in its entirety, has been developed and used for activity screening. Briefly, CD40 is coated at a preselected concentration, and binding of (FLAG-tagged) CD154 is measured by reacting with secondary antibody and using TMB liquid substrate system as a substrate for horseradish peroxidase for reading at 450 nm. The detailed procedure is as follows: Microtiter plates (Nunc F MaxiSorp) were coated with 1004/well of CD40: Fc (human:Fc human, recombinant from Alexis Biochemicals; MW=54 kDa) diluted in PBS pH 7.2 at a preselected concentration of 0.3125 μg/mL. Plates were covered with plate sealer and stored overnight at 4° C. The liquid was removed from the plates and blotted dry. Plates were blocked by washing once with approx. 300 μL/well of Blocking Solution (PBS pH 7.2, 0.05% Tween-20, 1% BSA) and incubated with the same solution for 1 hour at room temperature (RT) or at 4° C. overnight. Coated plates were washed three times with Washing Solution (PBS pH 7.4, 0.05% Tween-20) and blotted dry. A preselected dilution of CD154 (soluble human, FLAG-tagged, recombinant from Alexis Biochemicals; MW=18 kDa) of 0.0087500875 μg/mL range was prepared in 100 mM HEPES, 0.005% BSA pH 7.2. Binding was carried out by adding 100 μL of this CD154 dilution/well and incubating at RT in the presence of test compounds (or blank as negative control). After incubation, the plates were washed with Washing Solution and blotted dry. Bound CD154 was detected with 200 μL/well of secondary antibody mAb ANTI-FLAG M2—Peroxidase Conjugate (anti-FLAG M2-peroxidase HRP conjugate; Sigma-Aldrich) diluted 1:40,000 in Washing Solution. After incubation, the plates were washed and blotted dry. 200 μL/well of the HR-Peroxidase substrate TMB (3,3′,5,5′-tetramethylbenzidine; Sigma-Aldrich) were added, and the plates were incubated for approximately 30 minutes in the dark. The reaction was stopped with 0.5 M H₂SO₄ and read at 450 nm. All conditions were tested in at least three independent experiments in duplicate or triplicate per plates. Data were normalized and fitted with standard log inhibitor vs. response models using GraphPad Prism 5 to establish median inhibitory ICₛₒ values.

$$B = 100 \frac{C}{C + {IC}_{50}} = 100 \frac{1}{1 + {10}^{( log {IC}_{50} - log C )}}$$

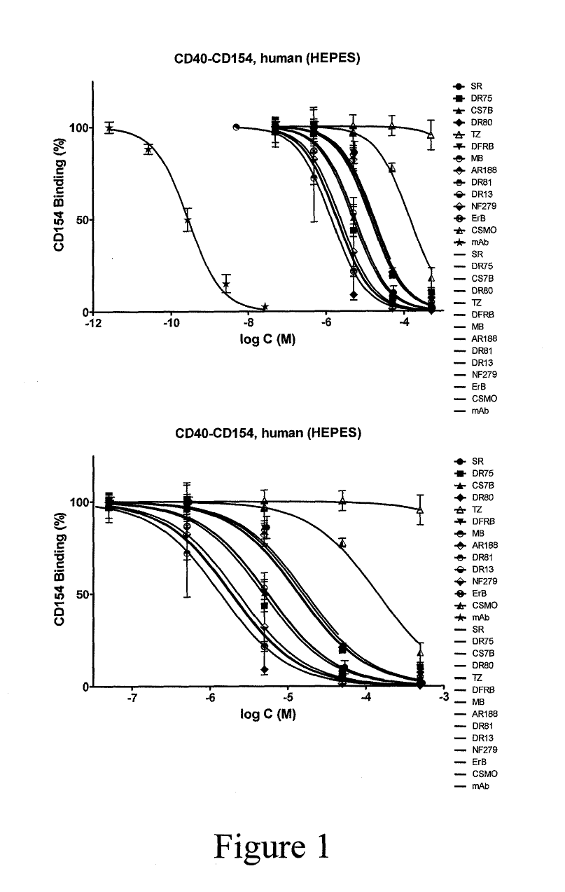

A representative result using the monoclonal anti-human CD154 antibody (R&D Systems, monoclonal anti-human CD40 ligand/TNFSF5 antibody, MAB617; MW≈150 kDa used) as positive control is shown in FIG. 1.

Binding inhibition assays with this system provided consistent and well-reproducible results, as FIG. 1 (bottom), focusing on the concentration range of their activities, illustrates. Some of the compounds tested [e.g., tartrazine, new coccine, or naphthol blue black] were essentially inactive (IC₅₀>1 mM), whereas some showed good activity with IC₅₀<10 μM (Table 1). The effect of DMSO, which was used as the diluting solvent for the non-water-soluble compounds and is a standard solvent in HTS assays, on the integrity of the assay has been examined. DMSO concentrations above 10% interfere with the binding assay, but concentrations below 3% have no effect. Hence, results obtained with DMSO dilutions less than 20-30-fold cannot be considered real; here, this only affects the highest concentrations tested for those compounds that required DMSO for dilution. Because 5 or 10 mM stock solutions were used, all IC₅₀ values below 250 μM can be considered as unaffected by DMSO.

### Example 2

### Murine CD40/CD154 Binding Inhibition Assay

Compounds of interest were also tested in a murine (mouse) model similar to the human one with murine CD40 and CD154 replacing the corresponding human proteins. This serves as additional confirmation of the in vitro inhibitory potential of these compounds, and it was considered important because mouse models are likely to be used as the first in vivo tests. Hence, the effectiveness of these compounds in inhibiting the mCD40:mCD154 interaction and not just the hCD40:hCD154 interaction was also verified. The experimental setup used was similar to the one described above, but using mCD40 (recombinant mouse CD40/TNFRSF5/Fc Chimera, soluble, R&D Systems) and (FLAG-tagged) mCD154 (recombinant mouse CD40L, soluble, Alexis Biochemicals). This time, however, a higher concentration of CD154 ligand had to be employed to obtain adequate signals, and after a number of calibration tests, mCD40 coating at 0.3125 μg/mL with mCD154 (mCD40L) at 1.1212 μg/mL range was selected to be used to screen the inhibiting activity of various compounds.

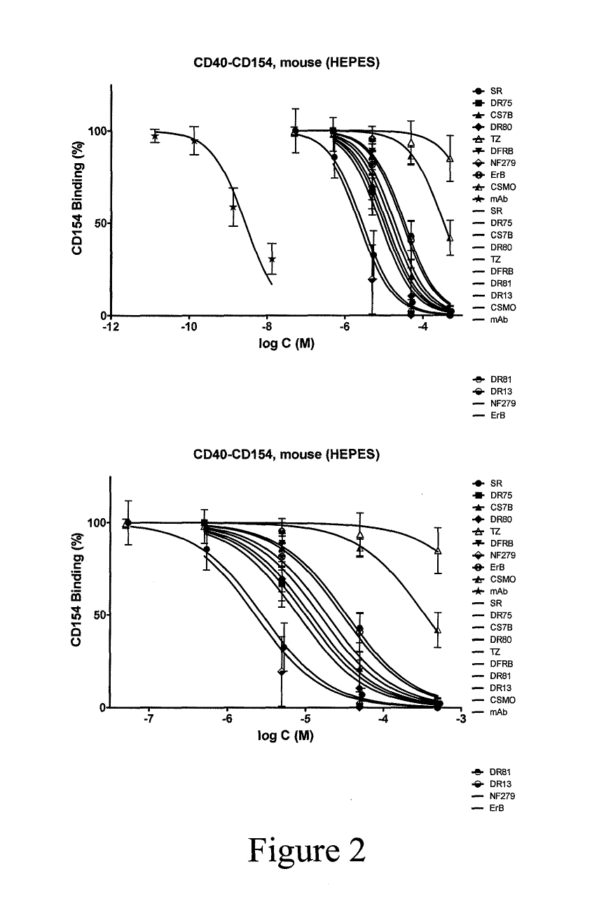

The experiments confirmed that the murine anti-CD154 monoclonal antibody (Taconic, MR1) was active in this system at the expected concentration (˜1 nM range), the human antibody was not, and vice versa, the human mAb was active in the human system, but not in the mouse system. The mouse system provided a similar range of activity with some (up to approximately five-fold) changes in activities compared to that seen in the human system (FIG. 2) (Table 1).

### Example 3

### Identification of the Binding Partner

It is also of interest to identify whether the compounds bind to the receptor CD40 or its ligand CD154 (CD40L). The main goal is to block the CD40/CD154 costimulatory interaction to achieve altered immune response and long-term tolerance; however, it seems more desirable to achieve this by targeting CD154, which is expressed mainly on activated T cells, since this way a more specific immune suppression could be achieved then by targeting CD40. To evaluate the binding partner of the test compounds, CD40 or CD154 were incubated for one hour with increasing concentrations of the test compounds, and after a wash, their ability to still bind their corresponding protein binding partner (CD154 or CD40, respectively) was assessed using a setup similar to that described before (Example 1).

### Binding to CD40

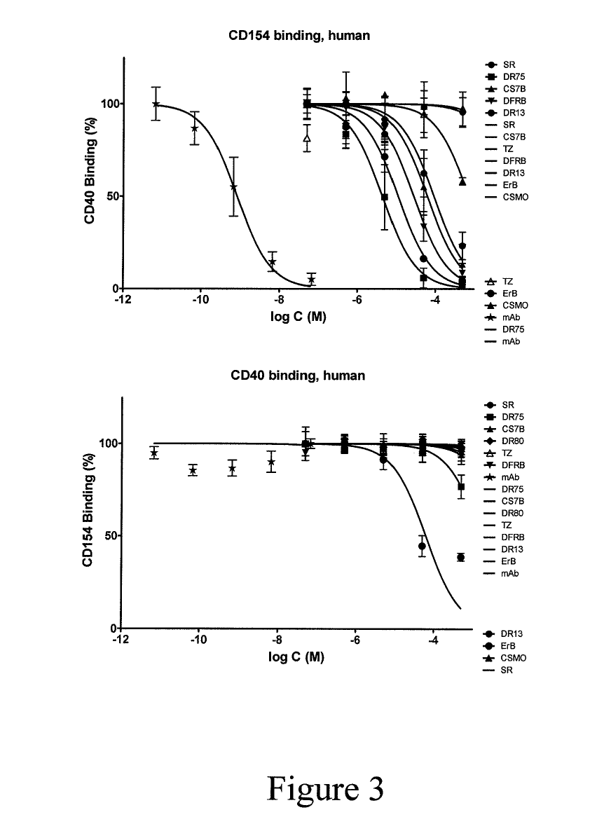

To test if compounds bind specifically to CD40, the procedure of Example 1 was followed with a modification. After blocking the plates with blocking solution and washing, 100 μL of different dilutions of the compounds were added to the wells and incubated at room temperature (RT) for 1 h to allow binding with CD40 in the absence of its CD154 ligand. The plates were then washed three times with washing solution. CD154 was then added and the amount bound was assessed as before. No significant binding was observed for any of the compounds tested with the exception of erythrosine B, which seems to be a ubiquitous binder (FIG. 3, top).

### Binding to CD154

For the reverse case, to test if compounds bind specifically to CD154, a similar procedure was followed, but the plates were coated not with CD40 but with CD154 (soluble, human Fc:CD40L, recombinant, Alexis Biochemicals), and binding of the soluble FLAG-tagged CD40:COMP (soluble, human CD40:COMP, recombinant, Alexis Biochemicals) was assessed in the final step.

The obtained signals indicate CD154 as the likely binding target: whereas for CD40, there was no significant inhibition for several compounds. The obtained IC₅₀ values for binding at CD154 were in general agreement with those obtained for their inhibitory activity (FIG. 3, bottom).

### Example 4

### Inhibition of the CD154-Induced Human B-Cell Proliferation Assay

CD40 stimulation is an important proliferation signal for human B cell proliferation (Fecteau et al., “CD40 stimulation of human peripheral B lymphocytes: distinct response from naive and memory cells. J. Immunol., vol. 171, pp. 4621-4629, 2003; Wiesner et al., “Conditional immortalization of human B cells by CD40 ligation,” PLoS ONE, vol. 3, pp. e1464, 2008), and soluble CD154 (CD40 ligand) can dose-dependently induce the proliferation of human CD19⁺ B cells as measured by standard proliferation .assays. To verify that this effect is inhibited by the present compounds, human CD19⁺ B cells were cultured in 96 wells tissue culture plates for 48 hours in the presence of CD154 and various concentrations of test compounds (including the anti-CD154 antibody as positive control), and then for another 48 hours in the presence of BrdU to asses proliferation and to compare it to negative and positive controls, respectively.

The detailed procedure is as follows: Frozen MPB CD19⁺ B lymphocytes (approximately 10⁶ cells per vial) were obtained from StemCells Technologies (Vancouver, Canada), thawed in a 37° C. water bath until no crystal piece was left, and the cell suspension was transferred to a 50 mL conical tube. The vial was rinsed with warm IMDM medium (Invitrogen) supplemented with 10% FBS (Invitrogen), 10 μg/mL of recombinant Human IL-4 (R&D Systems), 100 U/mL penicillin and streptomycin 100 μg/mL (Invitrogen) and IX of insulin-transferrin-selenium-G supplement (Invitrogen). Medium was slowly added to the cells while gently swirling the 50 mL conical tube until the total volume reach 15-20 mL, the cell suspension was centrifuged at 300 g and room temperature for 15 minutes, the supernatant was removed carefully without disturbing the pellet, and the cells were gently re-suspended in the remaining few milliliters of medium. This was repeated, the cells were counted, and the concentration of viable cells was adjusted to approximately 1×10⁶ cells/mL. Cells were cultivated in 96 wells flat-bottom tissue culture plates (Thomas Scientific). To activate them, CD154 (CD40L, soluble, human, recombinant, FLAG-tag from Alexis Biochemicals) and enhancer for ligands (Alexis Biochemicals) were added to the supplemented IMDM medium at final concentration of 0.11 μg/mL and 2 μg/mL, respectively. A colorimetric cell proliferation ELISA BrdU from Roche Applied Science (Indianapolis, Ind.) was used as immunoassay for quantification of cell proliferation based on the measurement of BrdU incorporation during DNA replication. Each experimental condition was tested in triplicate including the following controls i) culture medium plus BrdU (blank), ii) activated cells minus BrdU (background), iii) non activated cells plus BrdU, and iv) activated cells plus BrdU. Cells were cultured in the presence of various concentrations of test compounds in a final volume of 100 μL/well at a cell density of 5×10⁵ cells/mL. Commonly, this was obtained by adding 50 μL of 1×10⁶ cells/mL to each well and 50 μL of the test compound solution at twice the desired target concentration. Cells were incubated at 37° C., 90% humidity, and 5% CO₂, and parts of the samples were used to determine cell viability after 48 and 96 hours of cultivation using trypan blue staining on a hemacytometer. After 48 hours, 10 μL/well of BrdU labeling solution was added (prepared at 10-fold concentration in culture media from a 1000-fold concentrated stock), and cells were carefully re-suspend by pipetting and re-incubated for an additional 48 hours under the same conditions. After this, cells were re-suspended by pipetting, the plate was centrifuged at 300 g, and the supernatant was transferred to a clean plate and stored at −20° C. for further analysis. Cells were dried at 60° C., 200 μL/well of FixDenat was added, and the plate was incubated at room temperature. The FixDenat was removed by taping, and the plate was washed once with PBS+0.05% Tween-20 pH 7.4, and blocked with 200 μL/well of Blocking Solution (1% BSA, 0.05% Tween, PBS pH 7.4). The blocking solution was removed from the wells, they were washed with PBS+0.05% Tween-20 pH 7.4, and the plate was blotted dry. After this, 100 μL/well of anti-BrdU-POD working solution (monoclonal antibody from mouse-mouse hybrid cells, clone BMG 6H8, Fab fragment conjugated with peroxidase) was added and incubated for 30 minutes at room temperature. The antibody solution was removed, wells were washed with 200-300 μL/well of PBS+0.05% Tween-20 pH 7.4, and the plate was blotted dry. At this point, 100 μL/well of the HR-peroxidase substrate TMB were added, and the plates were incubated in the dark until color developed. The reaction was stopped with 1.0 M H₂SO₄ and read at 450 nm. Cursory cell viability evaluations were performed after 48 and 96 hours of cultivation using trypan blue staining on a hemacytometer.

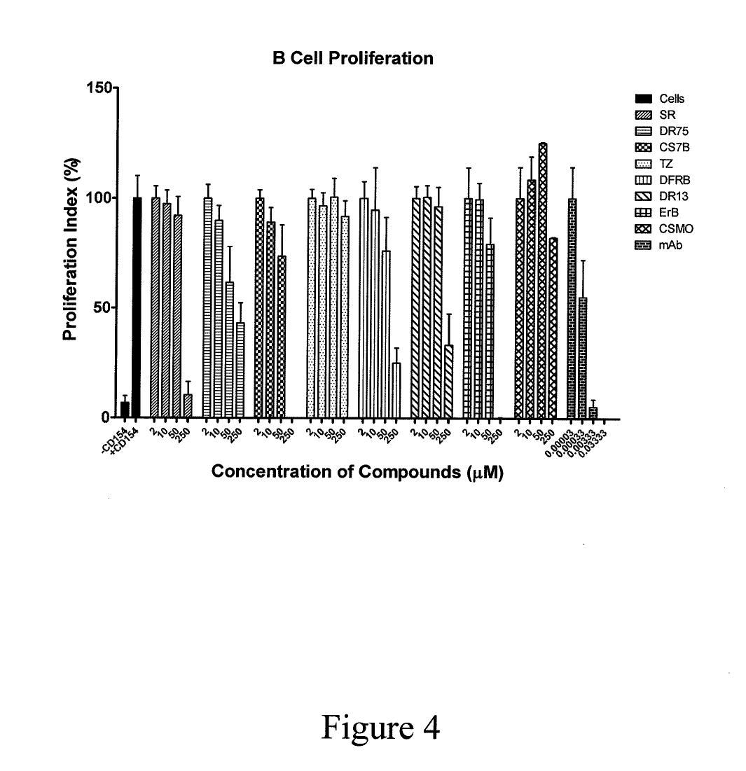

This assay confirmed that this effect is concentration-dependently inhibited by the corresponding mAb in the nM range, and also by some of the present compounds with IC₅₀s approximately in the same range as expected from the binding inhibition assays (FIG. 4) (50-100 μM for suramin, direct red 75, direct red 13, crocein scarlet 7B and direct fast red B). At these concentrations, the viability of cells was not significantly affected by any of these compounds. Tartrazine, a structurally related compound that was inactive in the binding assay, was used as a negative control, and it indeed had no proliferation inhibiting activity.

### Example 5

### Specificity Test—Human TNF-R1/TNFα Binding Inhibition Assay

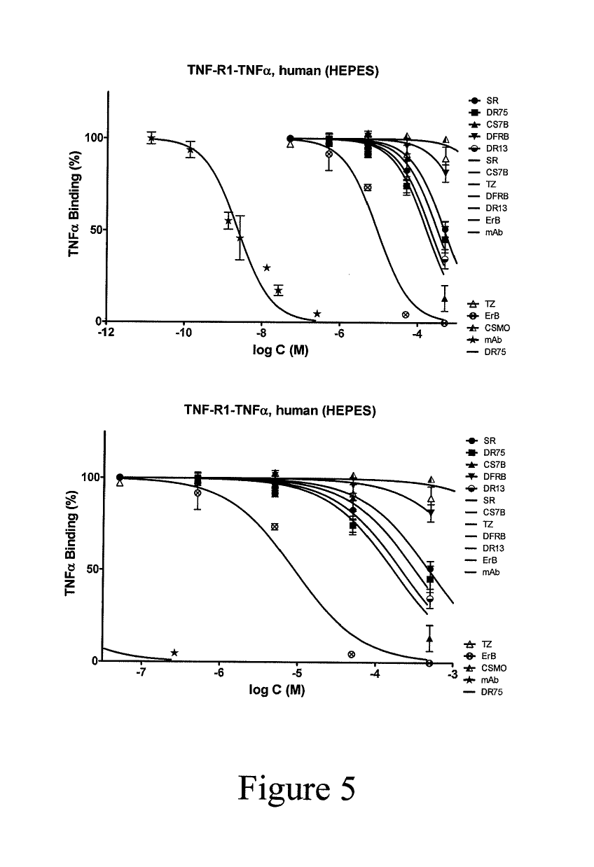

A main concern for the therapeutic applicability of these compounds and of small molecule PPI inhibitors in general is related to their ability to achieve some acceptable degree of selectivity/specificity for the PPI of interest. To identify compounds that show acceptable specificity for the CD40/CD154 system, their ability to inhibit the TNF-R1/TNF-α PPI was investigated since this is particularly relevant as CD154 is part of the TNF superfamily. The experimental setup used was very similar to the one described in Example 1, but using plate-coated TNF-R1 and TNF-α as ligand (with a corresponding anti-TNF-α mAb as positive control). The concentration of TNF-R1 and TNF-α used were 0.6 μg/mL and 0.02 μg/mL, respectively. Most compounds showed an acceptable degree of selectivity, inhibiting the binding of TNF-α to its receptor with an IC₅₀ of at least 30-40 fold higher than their IC₅₀ for the CD40/CD154 interaction, for example, direct red 13, direct fast red B, mordant brown 1, acid blue 29, and others (Table 1, FIG. 5). Among the compounds investigated, erythrosine seems a notable exception as it seems to show inhibitory activity in the 10 μM range for all interactions tested behaving as a nonspecific promiscuous inhibitor.

TABLE 1
Summary of obtained data for selected compounds

| | | Human | | | Mouse | | | |
|---|---|---|---|---|---|---|---|---|
| | | CD40/ | | | CD40/ | | Human | |
| | | CD154 | | | CD154 | | TNFR1/ | |
| | MW | log | IC₅₀ | IC₅₀ | log | IC₅₀ | TNFα | IC₅₀ |
| Compound | (salt) | IC₅₀ | (uM) | (ug/mL) | IC₅₀ | (uM) | log IC₅₀ | (uM) |
| mAb hu CD40L (R&D Syst, | 150000 | −9.56 | 0.0003 | 0.07 | >−5.0 | >10 | | |
| MAB617) | | | | | | | | |
| mAb mu CD40L (Taconic, | 150000 | >−5.0 | >10 | | −8.70 | 0.0020 | | |
| MR1) | | | | | | | | |
| Acid blue 113 | 681.7 | −5.95 | 1.1 | 0.8 | | | | |
| Acid blue 29 | 616.5 | −4.52 | 30.2 | 18.6 | | | −3.33 | 466.8 |
| Acid red 114 | 830.8 | −5.55 | 2.8 | 2.4 | | | −4.85 | 14.2 |
| Acid red 188 | 548.5 | −4.89 | 12.8 | 7.0 | | | | |
| Brilliant crocein (crocein | 556.5 | −3.85 | 140.9 | 92.8 | −3.55 | 282.5 | | |
| scarlet MOO) | | | | | | | | |
| Chlorazol black (direct black | 781.7 | −5.95 | 1.1 | 0.9 | | | | |
| 38) | | | | | | | | |
| Congo red | 696.7 | −5.24 | 5.7 | 4.0 | | | −4.46 | 34.9 |
| Crocein scarlet 7B | 584.5 | −5.26 | 5.5 | 3.6 | −4.86 | 13.7 | −3.77 | 168.3 |
| Direct blue 15 | 992.8 | −5.86 | 1.4 | 1.4 | | | | |
| Direct blue 71 | 1029.9 | −5.68 | 2.1 | 2.2 | | | −4.01 | 98.5 |
| Direct blue 120 (Pontamine | 877.8 | −5.80 | 1.6 | 1.4 | | | | |
| diazo blue BR) | | | | | | | | |
| Direct fast red B | 627.5 | −4.86 | 14.0 | 5.7 | −4.78 | 16.7 | −2.67 | 2162.7 |
| Direct red 13 | 712.7 | −5.26 | 5.5 | 3.5 | −4.90 | 12.7 | −3.67 | 214.5 |
| Direct red 37 | 676.6 | −4.88 | 13.0 | 8.8 | | | | |
| Direct red 53 | 663.6 | −4.61 | 24.4 | 16.2 | | | | |
| Direct red 75 (chlorazol fast | 990.8 | −5.32 | 4.8 | 4.9 | −5.13 | 7.4 | −3.49 | 322.1 |
| pink) | | | | | | | | |
| Direct red 80 | 1373.1 | −5.73 | 1.8 | 1.9 | −5.23 | 5.9 | −2.85 | 1428.9 |
| Direct red 81 | 675.6 | −4.87 | 13.6 | 6.9 | −4.47 | 33.8 | >−3.0 | >1000 |
| Direct yellow 27 | 662.6 | −5.72 | 1.9 | 1.3 | | | −4.37 | 42.4 |
| Erythrosin B (FD&C red 3) | 879.9 | −5.72 | 1.9 | 2.0 | −5.23 | 5.9 | −5.04 | 9.0 |
| Evans blue | 960.8 | −5.84 | 1.4 | 1.4 | | | | |
| Mordant brown 1 | 529.5 | −6.18 | 0.7 | 0.3 | | | −4.07 | 86.0 |
| NF279 | 1401.1 | −5.33 | 4.7 | 6.6 | −5.65 | 2.3 | −3.36 | 437.8 |
| Suramin | 1429.2 | −4.84 | 14.6 | 18.1 | −5.35 | 4.5 | −3.31 | 486.4 |
| Tartrazine (FD&C yellow 5) | 534.4 | >−3.0 | >2000 | >2000 | >−3.0 | >2000 | >−3.0 | >2000 |
| Trypan blue | 960.8 | −5.42 | 3.8 | 3.7 | | | | |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1-12. (canceled)

13. A method of inhibiting CD40/CD154 protein-protein interactions comprising exposing the proteins to an azo dye or suramin-related small molecule.

14. The method of claim 13, wherein said exposing step comprises administering an effective amount of an azo dye or suramin-related small molecule to a subject.

15. The method of claim 13, wherein said subject is afflicted with a disease or disorder involving the immune system.

16. The method of claim 13, wherein said exposing step comprises ex vivo culture of an organ or cell transplant in the presence of said azo dye or suramin-related small molecule before transplantation into a subject.

17. A method of treating a disease or disorder involving the immune system comprising administering an effective amount of an azo dye or suramin-related small molecule to a subject in need of treatment.

18. The method of claim 17 wherein the compound is selected from the group consisting of C.I. acid red 114, acid red 188, direct red 13, direct red 53, direct red 75, direct red 80, direct red 81, direct fast red B, crocein scarlet 7B, acid blue 29, acid blue 113, direct blue 15, direct blue 71, direct black 38, direct yellow 27, direct blue 120, Congo red, trypan blue, Evans blue, mordant brown 1, suramin, and NF279.

19. The method of claim 17 wherein the disease or disorder is selected from the group consisting of systemic lupus erythematosus (SLE), multiple sclerosis (MS), type 1 (juvenile) diabetes, rheumatoid arthritis, mixed connective tissue disease (MCTD), Celiac disease, Crohn's disease, ulcerative colitis, Grave's disease, Sjögren's syndrome, dermatomyositis, psoriasis, scleroderma, polymyositis, vasculitis, Wegener's granulomatosis, alopecia areata, chronic inflammatory diseases, autoimmune diseases, neurodegenerative disorders, graft-versus-host disease, cancer, atherosclerosis and the rejection of nonautologous organ or cell transplants.

20. The method of claim 17 wherein the disease or disorder is mediated through the CD40/CD154 pathway.

21. The method of claim 17 wherein the disease or disorder involves activation of immune responses mediated by T- and B-cells.

22-30. (canceled)

31. A pharmaceutical composition comprising an azo dye or suramin-related small molecule selected from the group consisting of C.I. acid red 114, acid red 188, direct red 13, direct red 53, direct red 80, direct red 81, direct fast red B, crocein scarlet 7B, acid blue 29, acid blue 113, direct blue 15, direct blue 71, direct black 38, direct yellow 27, direct blue 120, Congo red, trypan blue, Evans blue, mordant brown 1, and NF279; and a pharmaceutically acceptable carrier or excipient.

32. The method of claim 18 wherein the disease or disorder is selected from the group consisting of systemic lupus erythematosus (SLE), multiple sclerosis (MS), type 1 (juvenile) diabetes, rheumatoid arthritis, mixed connective tissue disease (MCTD), Celiac disease, Crohn's disease, ulcerative colitis, Grave's disease, Sjögren's syndrome, dermatomyositis, psoriasis, scleroderma, polymyositis, vasculitis, Wegener's granulomatosis, alopecia areata, chronic inflammatory diseases, autoimmune diseases, neurodegenerative disorders, graft-versus-host disease, cancer, atherosclerosis and the rejection of nonautologous organ or cell transplants.

33. The method of claim 18 wherein the disease or disorder is mediated through the CD40/CD154 pathway.

34. The method of claim 18 wherein the disease or disorder involves activation of immune responses mediated by T- and B-cells.
